(19) United States
(12) Patent Application Publication
(10) Pub. No.: US 2019/0290695 A1
(43) Pub. Date: Sep. 26, 2019

(54) MICRO-SLITS FOR RETICULOCYTE MATURATION

(71) Applicant: University of Rochester, Rochester, NY (US)

(72) Inventors: Richard Waugh, Pittsford, NY (US); Julia M. Kuebel, Rochester, NY (US); Yingye Fang, Urbana, IL (US); Greg Palis, Rochester, NY (US)

(21) Appl. No.: 16/302,734

(22) PCT Filed: May 19, 2017
(86) PCT No.: PCT/US2017/033490

§ 371 Date: Nov. 19, 2018

Related U.S. Application Data
(60) Provisional application No. 62/338,814, filed on May 19, 2016.

(51) Int. Cl. A61K 35/18; C12M 3/06; C12M 1/12; C12M 1/42; C12M 1/26

(52) U.S. Cl. CPC A61K 35/18; C12M 23/16; C12M 33/14; C12M 35/04; C12M 25/02

(57) ABSTRACT

The present disclosure relates to devices for producing mature red blood cells and methods for using the same. The devices comprise micro-slit filters that mechanically stimulate cells in custom cell medium to impart the cells with physical characteristics that enable the cells to pass through and survive human vasculature.

### CROSS-REFERENCE TO RELATED APPLICATIONS

This application claims the benefit of priority under 35 U.S.C. § 119(e) to U.S. Provisional Patent Application No. 62/338,814, filed May 19, 2016, the contents of which are incorporated by reference herein in its entirety.

### STATEMENT REGARDING FEDERALLY SPONSORED RESEARCH OR DEVELOPMENT

This invention was made with government support under grants UL1 TR000042, KL2 TR000095, and TL1 TR000096 awarded by the National Institutes of Health (NIH). The government has certain rights in the invention.

### BACKGROUND

Red blood cells (RBCs) are essential for human life. Blood lost due to trauma or anemia must be replaced to maintain the health of the individual. More than 16 million units of RBCs are transfused yearly in the United States, and the need for RBC therapy is projected to increase. In underdeveloped nations, the need for reliable blood supplies is even more acute. Risks of transmissible disease and immune reactions, while minimized, remain a serious threat to patients in need of chronic transfusion. Indeed, recent reports show that morbidity risk increases with the number of transfusions a patient receives, independent of other factors. The ability to produce made-to order red blood cells for transfusion would revolutionize transfusion medicine, and lead to substantial improvements in clinical care.

While several groups have reported the production of enucleated cells in erythroid cultures, only reticulocytes, and not fully mature RBCs, have been successfully transfused (Giarratana M C et al., Nature biotechnology 23.1 (2005): 69-74; England S J et al., Blood 117.9 (2011): 2708-2717; Giarratana M C et al., Blood 118.19 (2011): 5071-5079). Attempts to keep enucleated forms in culture results in their fragmentation and hemolysis (red cell death).

There is a need in the art for improved devices and methods for producing viable red blood cells.

### SUMMARY

Described herein is a micro-slit filter device for inducing that maturation of reticulocytes into mature RBCs, wherein the device comprises at least one micro-slit channel, each micro-slit channel comprising two openings connected by a lumen, wherein the two openings and the lumen comprises a width between about 1 and 3 μm.

In one embodiment, the micro-slit openings comprise a height between about 10 and 100 μm. In one embodiment, the lumen comprises a length between about 10 and 100 μm. In one embodiment, the micro-slits are spaced apart by a distance between about 5 and 50 μm. In one embodiment, the micro-slits comprise a taper. In one embodiment, the device is constructed from a material selected from the group consisting of: plastics, polymers, metals, glass, ceramics, silicon wafers, and polydimethylsiloxane (PDSM).

Also described herein is a microfluidic device comprising: a bottom substrate; a first layer positioned on top of the bottom substrate, the first layer comprising a fluid channel having at least one fluid port; a second layer positioned on top of the first layer, the second layer comprising a space fluidly connected to the fluid channel of the first layer; a third layer positioned on top of the second layer, the third layer comprising a space fluidly connected to the space of the second layer; a fourth layer positioned on top of the third layer, the fourth layer comprising a space fluidly connected to the space of the third layer; the micro-slit filter device described herein positioned within the space of the third layer; a top substrate positioned on top of the fourth layer, the top substrate comprising fluid tubes connected to the fluid ports of the first layer; and a compressible material surrounding the first layer, the second layer, the third layer, and the fourth layer, wherein the compressible material is sandwiched between the bottom substrate and the top substrate.

In one embodiment, the fourth layer comprises a compressible material. In one embodiment, the top substrate further comprises an open well fluidly connected to the space of the fourth layer. In one embodiment, the device further comprises between the first layer and the second layer: a fifth layer positioned on top of the first layer, the second layer comprising a space fluidly connected to the fluid channel of the first layer; a sixth layer positioned on top of the second layer, the third layer comprising a space fluidly connected to the space of the second layer; and a barrier filter positioned within the space of the sixth layer; wherein the barrier filter comprises micro pores 0.5 μm in diameter. In one embodiment, the device is subjected to ultraviolet/ozone treatment to increase hydrophilicity.

Also described herein is a method of mechanically stimulated culturing to produce red blood cells, the method comprising the steps of passing a volume of cell-containing medium through a micro-slit filter device comprising at least one micro-slit channel, each micro-slit channel comprising two openings connected by a lumen, wherein the two openings and the lumen comprises a width between about 1 and 3 μm.

In one embodiment, the cells are whole blood isolated CD71+ reticulocytes, whole blood isolated CD34+ reticulocytes, extensively self-renewing erythroblast (ESRE) derived reticulocytes, mesenchymal stromal cell-derived reticulocytes, bone marrow aspiration-derived reticulocytes, induced pluripotent stem cell-derived reticulocytes, embryonic stem cell-derived reticulocytes, and cord blood-derived CD34+ reticulocytes. In one embodiment, the mechanically stimulated culturing occurs at room temperature. In one embodiment, the mechanically stimulated culturing occurs at 37° C. In one embodiment, the cell-containing media is passed through the micro-slit filter device using the microfluidic device described herein. In one embodiment, the cell-containing media is passed through at least one micro-slit filter device in a bioreactor. In one embodiment, the cell-containing media is passed through the micro-slit filter device at a rate of 25 to 30 μL/min. In one embodiment, the cell-containing media is passed through the micro-slit filter device over a period of time between about 2 and 24 hours. In one embodiment, the cell-containing media is passed through the micro-slit filter device over a period of time between about 1 and 7 days. In one embodiment, the cell-containing media is passed through the micro-slit filter device once to leave the cells to culture within the micro-slit lumen.

### BRIEF DESCRIPTION OF THE DRAWINGS

The following detailed description will be better understood when read in conjunction with the appended drawings. For the purpose of illustrating the devices and methods described herein, there are shown in the drawings embodiments which are presently preferred. It should be understood, however, that the devices and methods described herein are not limited to the precise arrangements and instrumentalities of the embodiments shown in the drawings.

FIG. 1A depicts a diagram of reticulocytes passing through an exemplary micro-slit filter to form red blood cells. The integrated membrane filter comprises an array of slits that reproduce the dimension of the narrowest splenic sinus slits in vivo, ˜1-2 μm width.

FIG. 1B depicts a diagram of an exemplary microfluidic device comprising a micro-slit filter. This three-story assembly consists of (from bottom to top): a 1-mm thick standard glass slide (12,), a rubber cushion filler (14) between glass and polycarbonate enclosure (26), a 100-μm deep flow channel mounted on the glass (16), a 100-μm thick spacer between flow channel and barrier chip (18), a 300-μm thick frame for barrier chip (20), a 0.5-μm porous membrane-incorporated silicon chip (300-μm thick, 22), another 100-μm thick spacer between two silicon chips (18), another 300-μm thick frame for the slit-chip (20), a 1.5×50 μm slit-trap silicon chip (300-μm thick, 24), a 300-μm thick cushion layer (26) between the polycarbonate top-piece (5-mm thick, 28) and the slit chip.

FIG. 2A through FIG. 2C depict the assembly of an exemplary microfluidic device comprising a micro-slit filter. (FIG. 2A) A stack of three layers of polydimethylsiloxane (PDMS) gaskets: a U-shaped flow channel cut in the 100 μm thick gasket was mounted on a glass slide, a 100 μm thick spacer gasket with a 3×3 mm square cutout and two holes as flow inlets, and a 300 μm thick gasket with a 5.4×5.4 mm square cutout and two holes. (FIG. 2B) The barrier filter chip was embedded into the 5.4×5.4 mm square frame and mounted by another 100 μm thick spacer gasket. Lastly, another 5.4×5.4 mm 300 μm thick gasket was bonded to the spacer. Only the middle window of the barrier chip was aligned with the U-shaped channel. (FIG. 2C) The slit filter incorporated chip was placed into the frame and covered with a 300 μm thick gasket patterned with a 2×2 mm cutout and two holes for fluid passage.

FIG. 3A through FIG. 3C depict the experimental setup for a microfluidic device comprising a micro-slit filter. (FIG. 3A) Overview of all components. From top to bottom: a Y-shaped tubing for connecting the syringe pump to the microfluidic device, two 1/16″ female luer connectors, two binder clips, and a polycarbonate enclosure, ˜1 mm thick rubber padding, pre-bonded microchip. (FIG. 3B) The polycarbonate enclosure has a rectangular window serving as an open cell reservoir, and has two fluid conduits that aligned with the two 1.3 mm×1.3 mm holes on the PDMS gaskets to transport fluid from syringe pump to the bottom flow channel. (FIG. 3C) A close-up view of the pre-bonded microchip.

FIG. 4 is a schematic depicting the side profile of an exemplary microfluidic device comprising a micro-slit filter. The 1.2 mm thick microchip (a) constructed of 6 layers of PDMS gaskets and two silicon filter-integrated substrates was irreversibly mounted on a glass slide (b). A ˜1 mm thick rubber padding (c) was placed between the polycarbonate semi-closure (d) and the glass. This three-piece assembly was held together using two clamps (e). The ˜0.2-mm thickness difference between the microchip and the padding created an arch that allowed the assembly to be tightly sealed at the center.

FIG. 5 depicts an exemplary setup for controlling the flow of media to the microfluidic device comprising a micro-slit filter. Two pieces of PTFE tubing, one 10-cm long and the other 30-cm long with a split-end connecting to the two ports on the cell chamber, were used to transport medium from medium reservoir to syringe pump and from pump to cell chamber, respectively. There is a clamp on each split segment (I and II).

FIG. 6 depicts a portion of an exemplary interface for controlling an Arduino micro-controller system to drive the fluid in the microfluidic device. Parameters in the boxes are adjusted according to desired fluidic conditions.

FIG. 7 depicts an illustration for the Y-shaped tubing and its corresponding ports on the cell chamber.

FIG. 8 depicts the micropipette used to calculate measurements for cellular volume (V) and membrane area (A). Cells are aspirated with sufficient pressure to form a spherical portion outside the pipette and a cylindrical projection within the pipette. The outer diameter of the spherical part and the length of the projection are used to calculate area and volume from the given equations. Image is adapted from (Waugh R E et al., Experimental hematology 41.4 (2013): 398-408).

FIG. 9A and FIG. 9B depict a comparison of dimensional parameters of a fingerprick sample before and after the 48-hour incubation in a customized culture medium at 37° C. and 5% CO₂. (FIG. 9A) The sphericity value slightly increased after 48 hours of incubation in static medium. The majority of cells of the 48-hour-old sample appeared as normal discoid cells, and few were echinocytic (right). The scale bar on the picture is 8 um. (FIG. 9B) No significant difference observed in either surface area or volume after incubation. Statistical analysis was carried out using t test at 5% confidence level.

FIG. 10A and FIG. 10B depict the results of experiments demonstrating increases in circulating CD71+ reticulocyte sphericity during 48-hour post-isolation static culture at different incubation temperatures. (FIG. 10A) The blue bar represents the mean sphericity of fresh red blood cells. There is no significant difference between the green and yellow bars, which represent the mean sphericity of fresh and 24-hour-old reticulocytes incubated at 4° C., respectively. There is a significant leap from 24-hour-old reticulocytes incubated at 4° C. and same age lineage incubated 37° C. (yellow vs. red). (FIG. 10B) Further analysis for FIG. 10A by breaking the dimensionless sphericity (of the blue, green, and red columns) into membrane area and volume. 50 cells were measured for each column, error bars are one standard deviation (SD). Statistical significances were indicated by t test at 5% confidence level.

FIG. 11A and FIG. 11B depict a comparison of mean sphericity values, membrane area, and cellular volume of a finger-prick sample with and without a 4-hour ‘massage’ at room temperature. (FIG. 11A) Sphericity values were compared between massaged and statically-incubated cells at room temperature, p<0.05. On the other hand, (FIG. 11B) the difference in neither membrane area nor cellular volume is significant (t test at 5% confidence level). 35 cells were measured for each column, error bars are one standard deviation (SD).

FIG. 12 depicts sphericity measurements before and after 2 or 4 hr of microfluidic treatments. 50 cells were measured for each column, error bars are one standard deviation. Stars designate a significant difference between groups (t-test, p <0.05).

FIG. 13A through FIG. 13C depict further examination of the results in FIG. 12 by breaking down the dimensionless sphericity number into membrane area and volume. (FIG. 13A) There is a slight decrease in the mean cellular volume observed after the microfluidic treatment, but it is not significant based on the t-test at 5% confidence level. (FIG. 13B, FIG. 13C) The decrease in volume after the microfluidic treatment is significant based on the t-test at 5% confidence level. 50 cells were measured for each column, error bars are one standard deviation (SD).

### DETAILED DESCRIPTION

Described herein are devices and methods for producing mature red blood cells and methods for using the same. The devices comprise one or more micro-slit filters that mechanically stimulate cells to impart the cells with physical characteristics that enable the cells to pass through and survive human vasculature.

### Definitions

It is to be understood that the figures and descriptions of the present disclosure have been simplified to illustrate elements that are relevant for a clear understanding of the present disclosure, while eliminating, for the purpose of clarity, many other elements typically found in the art. Those of ordinary skill in the art may recognize that other elements and/or steps are desirable and/or required in implementing the devices and methods described herein. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The term “sphericity” refers to a dimensionless value of a cell's volume to membrane surface area ratio. A cell's sphericity is a measure of its deformability.

Throughout this disclosure, various aspects can be presented in a range format. It should be understood that the description in range format is merely for convenience and brevity and should not be construed as an inflexible limitation on the scope of the presently described devices and methods. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

### Description

Splenic sinuses of the red pulps, also known as sinusoids, mechanically filter mature RBCs as they enter the spleen. Human RBCs (˜8 μm diameter) leaving the blood vessels in the spleen's red pulp must essentially find their own way back into circulation by squeezing through slits in the walls of the sinusoids (˜1-2 μm in width). If the RBC has any inflexibility or any abnormal granules, it is removed from circulation and devoured by nearby macrophages. During steady-state erythropoiesis in vivo, the splenic sinusoid is commonly believed to also play a major part in the mechanical remodeling process. R1 reticulocytes are multi-lobular and active in endocytosis. They mature into R2 reticulocytes, which are more regular in contour and contain 1-2 autophagic inclusions per cell, and soon enter the peripheral circulation. Intracellular vacuoles release cytoplasmic contents through exocytosis after numbers of passages through splenic sinus slits (solid red arrow). It was also reported that, exosomes (extracellular vesicles) were found on the reticulocytes both in-vivo and in-vitro. It is believed that these blebs are either released from the cells when multivesicular bodies fuse with the plasma membrane or they are released directly from the plasma membrane during the continued traversing in the spleens. At the end of the maturational process, detached vesicles are found in the spleen and engulfed by macrophages. The result of this process is the generation of biconcave organelle-free CD71-negative erythrocytes possessing only ˜20% less membrane area compared to the R1 reticulocytes.

Described herein are in vitro and ex vivo devices and methods for inducing the maturation of blood cell precursor cells into mature RBCs. For example, in various embodiments, the devices and methods described herein utilize one or more micro-slit channels or filters having a width of about 1-3 μm, wherein precursor cells are mechanically deformed within the micro-slit channels. Mechanical deformation within the micro-slit channels induces the maturation of the precursor cells into enucleated mature RBCs. The mature RBCs may be used in a wide variety of clinical applications, including, but not limited to use in surgical, traumatic, emergency medicine, or personalized medicine settings.

### Micro-Slit Filter Device

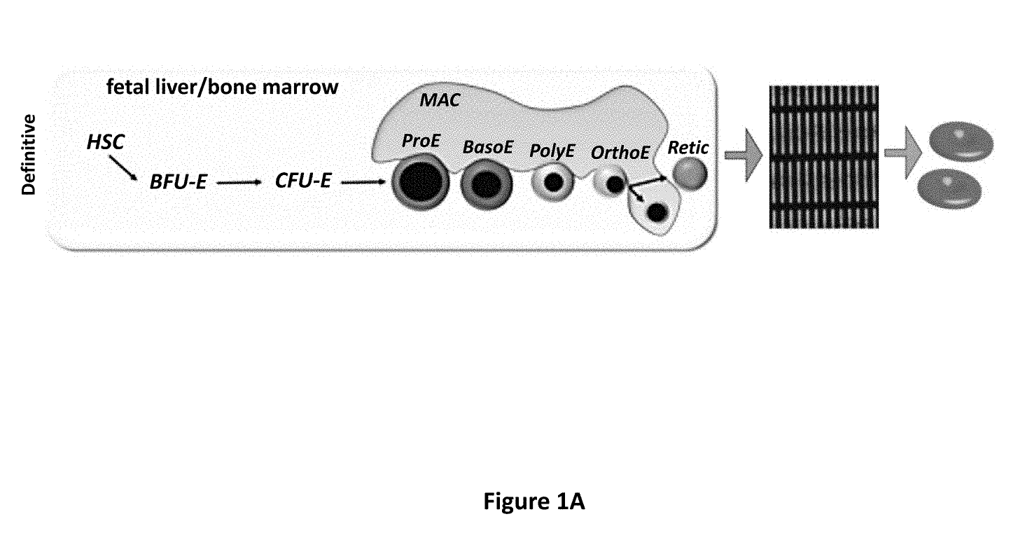

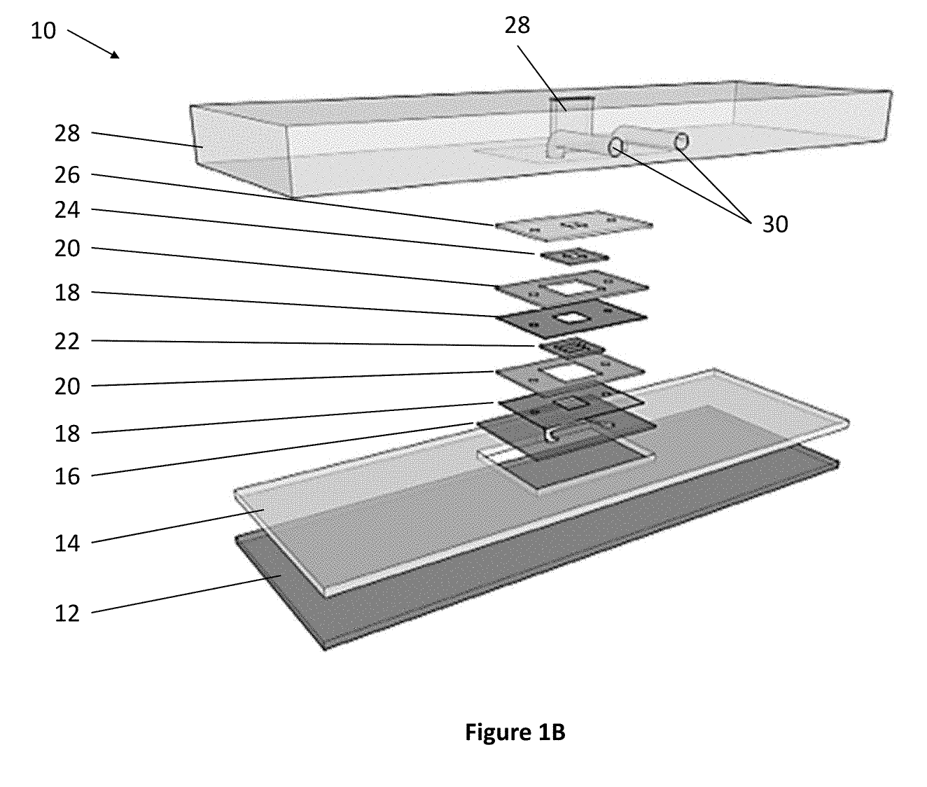

Described herein are micro-slit filter devices for inducing the maturation of reticulocytes into mature RBCs. An exemplary micro-slit filter comprises an array of at least one micro-slit, such as the array depicted in FIG. 1A. The micro-slits are substantially rectangular channels having two openings connected by a linear lumen. The micro-slits comprise a height, a width, and a length, wherein the height and width describe the dimensions of the two openings, and the length describes the distance between the two openings.

The micro-slits can be unidirectional or bidirectional. In some embodiments, the openings and the lumen comprise a constant geometry and shape throughout. In some embodiments, the openings and the lumen are tapered. For example, one opening may be larger than the other in height, width, or both, and the lumen may taper from the larger opening to the smaller opening. A tapered micro-slit may be advantageous for unidirectional use, wherein the larger opening can more easily capture cells to be pushed out of the smaller opening.

In some embodiments, both openings may have the same geometry and shape, and the lumen tapers to a narrower geometry between the two openings. A micro-slit that is narrower in the lumen than in both openings may be advantageous for bidirectional use, wherein the two larger openings can more easily capture cells from either direction, and the narrower lumen provides the exact desired mechanical stimulation.

In various embodiments, the width of the micro-slits can be between about 1 and 3 μm. In certain embodiments, the width of the micro-slits is preferably between about 1.5 and 2 μm. In certain embodiments, the width of the micro-slits is preferably between about 1.8 and 2 μm. In certain embodiments, the width of the micro-slits is preferably between about 1.5 and 1.8 μm. The height of the micro-slits can be any height, such as a height between about 10 and 100 μm. The length of the micro-slits can be any length, such as a length between about 10 and 100 μm. The micro-slits may be spaced apart by any distance, such as a distance between about 5 and 50 μm.

The micro-slit filter device can be constructed from any suitable material. Non-limiting examples include plastics, polymers, metals, glass, ceramics, and the like. In some embodiments, preferable materials include silicon wafers and polydimethylsiloxane (PDSM).

### Microfluidic Device

Described herein are microfluidic devices for applying mechanical stimulation to cells in culture, for example to induce the maturation of reticulocytes to mature RBCs. Referring now to FIG. 1B, an exemplary microfluidic device 10 is depicted. In certain embodiments, microfluidic device 10 comprises a plurality of layers, wherein the layers consist of substrate 12, cushion filler 14, deep flow channel 16, spacer 18, frame 20, barrier filter 22, micro-slit filter 24 (as described elsewhere herein), cushion layer 26, and top piece 28.

Substrate 12 can be any suitable substrate capable of providing microfluidic device 10 with structural rigidity. Non-limiting examples of suitable substrate materials include glass, plastic, metal, ceramic, wafers, curable polymers (e.g., PDMS) and the like. In some embodiments, substrate 12 can be a glass slide.

Cushion 14 provides microfluidic device 10 with at least some semi-compressibility. In some embodiments, cushion 14 prevents fluid from leaking out of microfluidic device 10. For example, in certain embodiments, the components of the device are compacted together, and cushion 14 provides a watertight seal around the perimeter of the device. Cushion 14 can be any suitable semi-compressible material, including but not limited to natural and synthetic polymers such as rubber and silicon.

Deep flow channel 16 consists of a planar structure having a channel with a port at both ends for inflow/outflow of fluid. Deep flow channel 16 can be constructed from any suitable material, such as glass, plastic, metal, ceramic, and the like.

Spacer 18 consists of a planar structure having at least one space within the structure to permit the passage of fluid. At least one spacer 18 is provided to maintain clearance between certain layers of microfluidic device 10. The clearance is preferable to provide a space for fluid to flow through. In certain embodiments, the clearance provides a space for cells to collect. Spacer 18 can be constructed from any suitable material, such as glass, plastic, metal, ceramic, and the like.

Frame 20 consists of a planar structure having at least one space within the structure for accepting smaller components and holding them in place, such as the filters described herein. Frame 20 can be constructed from any suitable material, such as glass, plastic, metal, ceramic, and the like.

Barrier filter 22 comprises a filter having pores smaller than the diameter of any cultured cells, such as pores having a diameter of 0.1 to 0.5 μm. Barrier filter 22 is provided to prevent cells from being lost during outflow of culture media.

Cushion layer 26 consists of a planar structure having at least one space within the structure to permit the passage of fluid. In some embodiments, cushion layer 26 prevents fluid from leaking out of microfluidic device 10. For example, in certain embodiments, the components of the device are compacted together, and cushion layer 26 provides a watertight seal at the top of the device. Cushion layer 26 can be any suitable semi-compressible material, including but not limited to natural and synthetic polymers such as rubber and silicon.

Top piece 28 consists of a rigid block structure to fit on top of the layers of the microfluidic device. In various embodiments, the layers of the microfluidic device are compressed between top piece 28 and substrate 12. In some embodiments, top piece 28 comprises tubes 30 that are fluidly connected to the ports of deep flow channel 16 to permit the inflow and outflow of fluid. In some embodiments, top piece 28 comprises well 32 for loading cell-containing media, and for accepting overflow of added fluid. In certain embodiments, well 32 is open to the atmosphere. In other embodiments, well 32 may be closed with an air-permeable cover.

In some embodiments, the microfluidic devices are scalable for larger output of mature RBCs. For example, the microfluidic devices can be scaled up to industrial-sized bioreactors with larger, deeper reservoirs for holding cell-containing culture medium. To increase output, a typical RBC-producing bioreactor may comprise a plurality of micro-slit filters, or larger filters comprising the micro-slit arrays described herein. Larger filters having diameters in the range of 4-12 inches are capable of filtering a much larger volume of liquid. Larger filters are also capable of filtering liquids having higher cell concentrations. A bioreactor may also obviate the use of a barrier filter. For example, the flow of media and cells within a bioreactor may be in a single directional flow, wherein the media and cells circulate through one or more micro-slit filter.

### Methods of Making

The devices described herein can be made using any suitable method known in the art. The method of making may vary depending on the materials used. For example, components substantially comprising a plastic or polymer may be milled from a larger block or injection molded. Likewise, components substantially comprising a metal may be milled, cast, etched, or deposited by techniques such as vapor deposition, spraying, sputtering, and ion plating. In some embodiments, the devices may be made using 3D printing techniques commonly used in the art. Microstructures, such as the micro-slit filters, may be fabricated using photolithography, vapor deposition, etching, and micro-forming.

In certain embodiments, it may be advantageous to treat the layers of the microfluidic devices to better prevent fluid leakage. For example, the layers may be bonded using sealants, or covalently bonded using ultraviolet/ozone treatment. Bonding may be enhanced through additional curing steps. Microfluidics may be improved by reducing the likelihood of introducing air into the microfluidic device. For example, the microfluidic device may be exposed to ultraviolet/ozone treatment or any other suitable treatment to yield hydrophilic surfaces.

### Methods of Making Mature Red Blood Cells

Described herein are methods of making mature red blood cells through mechanical stimulation and culture in custom media. The methods include steps for loading the microfluidic devices described herein and adjusting flow parameters.

Prior to introducing cells into a microfluidic device, the microfluidic device is first prepared to minimize the formation of air bubbles. In certain embodiments, the microfluidic device may be subjected to a final ultraviolet/ozone treatment to increase hydrophilicity. The microfluidic device is flushed with culture medium several times to void air and to coat the inner surface. The flushing step may be performed manually with a syringe, or automatically with a pump. The microfluidic device is then loaded fully with culture medium and allowed a period of time for the culture medium to fully saturate the inner surfaces and filter spaces of the device.

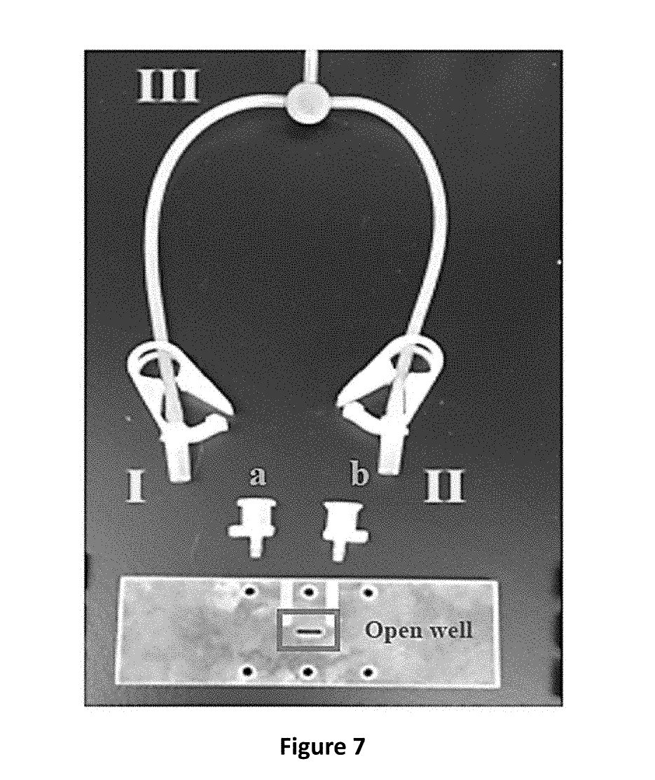

After the microfluidic device has been prepared, a volume of loaded culture medium is withdrawn and a volume of cell-containing culture medium is inserted into the microfluidic device, such as in the open well depicted in FIG. 7.

The cells may be any population of cells amenable for maturation to RBCs, including but not limited to, whole blood isolated reticulocytes (e.g., CD71+ reticulocytes, CD34+ reticulocytes), extensively self-renewing erythroblast (ESRE) derived reticulocytes, mesenchymal stromal cell-derived reticulocytes, bone marrow aspiration-derived reticulocytes, induced pluripotent stem cell-derived reticulocytes, embryonic stem cell-derived reticulocytes, cord blood-derived reticulocytes (e.g., CD34+ reticulocytes), isolated bone marrow-derived hematopoietic stem cells, other isolated hematopoietic cells, other erythrocyte precursor cells, and the like. Isolated bone marrow-derived hematopoietic stem cells may be collected and isolated by means known in the art. Other precursor cells may also be isolated by means known in the art, for example using flow cytometry or other sorting methods for hematopoietic cells or other erythrocyte precursor cells using commonly used markers. In some embodiments, precursor cells are differentiated in culture using means known in the art such as through use of media supplements directing precursor cells towards an erythrocyte or reticulocyte fate prior to being loaded into the device.

After loading the cells, mechanically stimulated culturing of the cells is carried out. Culture media is drawn in and out of the microfluidic device to pass the cell-containing culture media through the micro-slit filter. In some embodiments, the rate of culture media flow is between about 25 and 30 μL/min. In some embodiments, the pattern of flow cycling between drawing culture medium in and out for about 30-60 seconds per draw with a 10 second period of rest in between drawing in or out. The flow of liquid may be performed manually, or it may be controlled using a computer platform, such as an Arduino micro-controller system.

In some embodiments, the mechanically stimulated culturing of the cells may be performed at room temperature. Room temperature is typically a temperature in a range between about 18 and 24° C. In some embodiments, the mechanically stimulated culturing of the cells may be performed at or around 37° C. The length of mechanically stimulated culturing time can be any suitable time. For example, in some embodiments, the cells may be cultured for a period of time between about 2 and 6 hours. In some embodiments, the cells may be cultured for a period of time between about 24 and 72 hours. In some embodiments, the cells may be cultured for up to 4 days or longer.

In some embodiments, the mechanically stimulated culturing of the cells may be performed without cycling the culture media. For example, in some embodiments wherein a micro-slit filter comprises micro-slits having lengths long enough to accommodate multiple cells, cells may be drawn into the micro-slits using a single drawing in cycle, then left to culture within the micro-slits for any suitable duration.

In certain embodiments, the method comprises the use of culture medium that is able to provide cells with sufficient energy to respond to mechanical stimulus while maintaining their viability and physiological properties during mechanical stimulation. In some embodiments, the medium is conditioned medium. In some embodiments, the conditioned medium is medium isolated from a culture of cells and is subsequently used to culture cells under mechanical stimulation. In some embodiments, the medium is conditioned by other means known in the art. In certain embodiments, the medium is resistant to bubble formation to avoid disrupting the operation of the micro-slit device.

In some embodiments, the base of the medium is a bicarbonate-free medium, such as Dulbecco's Modified Eagle Medium (DMEM-D 1152), to discourage bubble forming. In some embodiments, the basal medium is any suitable basal media known in the art. In some embodiments, the medium is supplemented with raised glucose levels (4500 mg/L, 25 mM) and/or HEPES, wherein the HEPES concentration is about 25 mM. A high glucose level is beneficial for maintaining physiological homeostasis of maturing cells.

In some embodiments, the osmolarity of the medium is adjusted. For example, to match normal plasma osmolarity (-290-300 mosmol/kg), an additional 122 mg of glucose and 40 mg of sodium chloride can be added for every 25 ml of medium. In certain embodiments, the medium is further treated just prior to use in a microfluidic device. Further treatment steps include the addition of 4% Fetal Bovine Serum (FBS, Gemini #900108) and 1% penicillin/streptomycin (Sigma #P4333), followed by filtration and de-gassing in vacuum. In some embodiments, less than 4% FBS is added. In some embodiments, at least about 4% FBS is added. In some embodiments, less than about 1% to no penicillin/streptomycin is added. In some embodiments, the osmolarity of the medium is decreased by lowering salt concentration. A lower salt concentration leads to less deformation and rounder cell geometry, and may be beneficial for early reticulocyte development.

The medium may be supplemented with one or more additional agents that help support the survival, differentiation, proliferation, or maturation of the cells. For example, in certain embodiments, the medium is supplemented with one or more growth factors, hormones, antibiotics, anticoagulants, vitamins, or the like. Exemplary agents include, but are not limited to hematopoietic growth factors (HGF) such as erythropoietin (EPO), granulocyte colony-stimulating factor (G-CSF), granulocyte-macrophage colony-stimulating factor (GM-CSF), macrophage CSF (M-CSF), IL-3, thrombopoietin (TPO), stem cell factors, antioxidants, and vitamins such as vitamin E.

In certain embodiments, the cells are modified. For example, in certain embodiments, the cells are genetically modified to express one or more proteins of interest. For example, the cells may be modified to express a therapeutic protein that aids in the survival, differentiation, proliferation, or maturation of the cells. In certain embodiments, the cells are modified to introduce a mutation in the cells or to correct a mutation in the cells. Cells may be modified using any known technique known in the art.

In certain embodiments, the cells are contacted with one or more agents during the maturation of the cells. For example, the cells may be contacted with a small molecule, peptide, antibody, nucleic acid molecule, or the like. In certain embodiments, the cells may be contacted with an inhibitory agent to inhibit the activity or expression of one more proteins.

In certain embodiments, the device and method described herein can be used to screen one or more agents or compounds. For example, in certain embodiments, one or more candidate compounds (e.g., from a compound library) can be administered to the cell or medium to evaluate the effect of the one or more candidate compounds on cell survival, differentiation, proliferation, or maturation.

RBCs matured by way of the devices and methods described herein are useful for applications such as blood infusions and blood replacement. Patients who have blood loss from trauma, a blood cell disease, a blood cell deficiency, or any other related disease or disorder may benefit from the presently described devices and methods.

In some embodiments, a patient's own precursor cells may be isolated, matured by way of the devices or methods described herein, and transplanted back into the patient as appropriate. In some embodiments, donor precursor cells cultured in vitro or isolated from a subject may be matured by way of the devices or methods described herein and transplanted into a patient as appropriate.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the claimed devices and practice the claimed methods. The following working examples therefore, specifically point out the preferred embodiments, and are not to be construed as limiting in any way the remainder of the disclosure.

### Example 1: Mechanically-Induced Membrane Remodeling During Reticulocyte Maturation in a Microfluidic Device

The complex process of erythropoiesis can be divided into three progressive phases: (1) progenitor expansion, (2) precursor amplification and maturation, and (3) reticulocyte remodeling into terminal erythrocytes. The first two phases have been extensively explored by many research groups worldwide, and significant achievements in differentiating hematopoietic stem cells into enucleated reticulocytes in vitro have been reported. It is known that during the third phase, the reticulocyte must lose ˜20% of its surface area, reduce its volume, and degrade or eliminate residual cytosolic organelles (Mankelow T J et al., Blood 126.15 (2015): 1831-1834). Many current studies are consistent with the concept that loss of plasma membrane is through the release of endocytosed plasma membrane as exosomes (Griffiths R E et al., Blood 119.26 (2012): 6296-6306).

Certain external stresses exerted on reticulocyte membranes before the exocytosis of membrane proteins plays a critical role in retaining a sufficient excess of membrane to allow mature erythrocytes to change shape and always enclose the hemoglobin-rich cell contents in the narrow microcirculation. The present study examines a mechanical micro-system that is able to apply external forces that imitate the shear stress in the microcirculation and splenic slits to produce reticulocytes in vitro.

The materials and methods are now described.

Enrichment of Circulating CD71+ Reticulocytes from Whole Blood

Reticulocytes are enucleated immature red blood cells, typically comprising about 1-2% of the red cells in the human body. Young reticulocytes are characterized by a high expression of transferrin receptor (CD71), a reticulocyte-specific surface protein that disappears during late-stage maturation in the circulation as the cell fully matures into an erythrocyte. Thus, reticulocytes newly released from bone marrow can be identified and isolated from whole blood using anti-transferrin antibody. A conventional approach used in many studies for isolating pure young reticulocytes from peripheral blood involves the use of immunomagnetic beads. The well documented positive selection of human reticulocytes by immunomagnetic separation method was adopted and slightly modified for this work (Brun A et al., Blood 76.11 (1990): 2397-2403).

However, as the process of reticulocyte isolation normally required at least 4 hours, including 1-1.5 hours of blood filtration, 2-4 hours of incubation, at least 1 hour of cell wash, plus other procedures, it was not realistic to complete the reticulocyte isolation, the microfluidic testing, and the subsequent sphericity measurements in one day. A complicating factor was that reticulocytes get smaller quickly in vitro. Gronowitz et al. stated that 60% of the cells were spherical or spheroechinocytic at 48 hours when maturation is complete (Gronowicz Get al., Journal of cell science 71.1 (1984): 177-197). Previous results of scanning EM also suggested that small pieces of membrane were lost during in vitro incubation, which might cause the formation of spherocytes rather than normal biconcave disks (Noble N A et al., Blood 74.1 (1989): 475-481). These results supported the hypothesis that, in the absence of mechanical stress, reticulocytes do not undergo proper membrane remodeling. As a result, they lose more membrane area and become less deformable compared to natural erythrocytes.

It was demonstrated by prior studies that reticulocytes do not mature at 4° C. in vitro for 2 weeks after they were drawn from animals (Golenda C F et al., Proceedings of the National Academy of Sciences 94.13 (1997): 6786-6791). Sphericity measurements for reticulocytes before and after storage at 4° C., room temperature, and 37° C. were taken and compared. The results also provide evidence that isolated reticulocytes can be stored at 4° C. for 24 hours without significant changes in their area and volume, and therefore, would be suitable for microfluidic testing.

Procedure Planning

To examine the effect of the microfluidic system on mechanically decreasing the volume-to-surface area ratio of CD71+ reticulocytes, experiments were replicated three times using CD71+ reticulocytes from three donors. Each experiment consisted of (1) the construction of the microfluidic device; (2) the preparation of microfluidic culture medium; (3) reticulocyte isolation from venous whole blood using anti-CD71 magnetic beads; (4) setting and testing flow parameters (flow rate, duration per cycle, etc.) via the Arduino micro-controller; (5) a 2-4 hour microfluidic treatment continuously monitored under an inverted microscope (10-40×) at room temperature; and (6) measurements of sphericity for ‘massaged’ and ‘unmassaged’ reticulocytes using a ˜1.5 μm diameter micropipette under 15-cm H2O pressure, >50 cells/sample were measured. Steps (1) and (2), step (3), and steps (4)-(6) were conducted on Day 1, Day 2, and Day 3, respectively.

Construction of Microfluidic Device

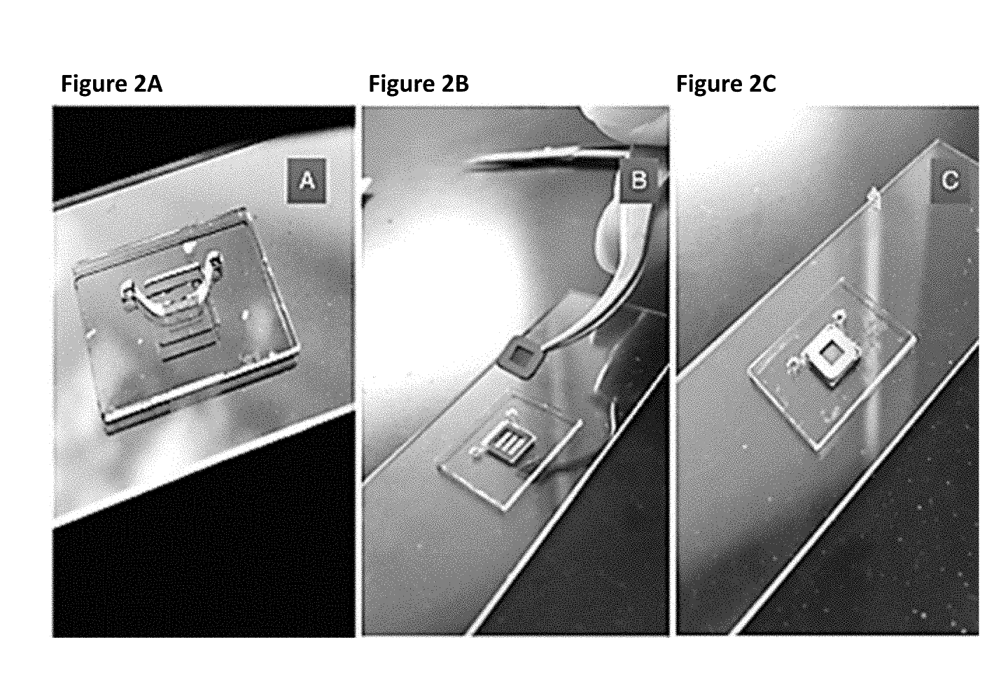

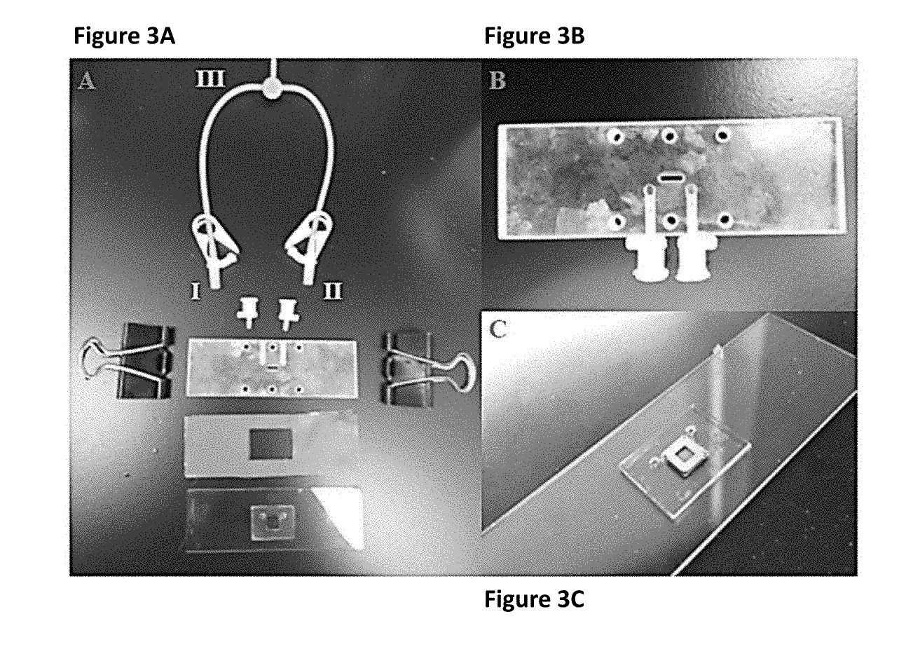

Each multilayered microfluidic chip was constructed of 6 layers of PDMS gasket sheets of varied thickness and layouts by covalent bonding using ultraviolet/ozone treatment (7 mins for each cycle). The fluid space was divided into three compartments by two silicon filters (FIG. 2A through FIG. 2C). The lower filter was a 2 mm×1 mm thin silicon film, the pattern of which was an array of 0.5 μm diam circular pores, and the upper filter was a 5 mm×5 mm 10 μm thick silicon film patterned with an array of 1.5×50 μm slit traps. Two 1.5×1.5 mm holes patterned into all the gaskets above the bottom U-shaped flow channel were carefully aligned with entrances to the fluid conduits on the top-piece (FIG. 3A, FIG. 3B). A 2-hour curing process at ˜60° C. ensured the effective bonding between layers. Desired gasket geometries were precisely created using the Silhouette CAMEO cutter (Silhouette America, Oren, Utah).

A top-piece structure, which is a 5 mm thick transparent polycarbonate cover that has a 2 mm×1 mm open window that vertically aligned with the filters and has two elbow-shaped fluid conduits that linked the bottom flow channel to the syringe pump, was placed on the microchip and held by two binder clips (FIG. 3A). Cells were fed through the open window and went back and forth across the upper filter with the in/outflow in the bottom flow channel. The PDMS-based microchip was constructed shortly before use, and the polycarbonate top-piece was a reusable piece.

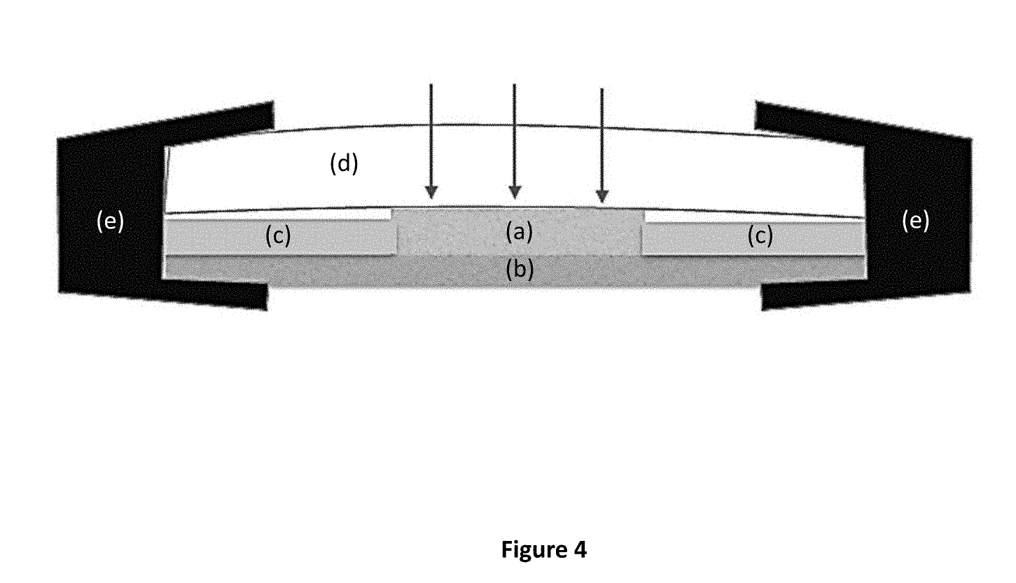

The thickness of rubber padding (˜1 mm) was somewhat critical. It should be slightly thinner than the bonded PDMS stack (1.2 mm) so that a compressing pressure was created between the polycarbonate cover and the microfluidic chip when clamped together with two binder clips (FIG. 4).

Microfluidic Culture Medium Preparation

The culture medium in which the cells are suspended must be able to provide cells energy to respond mechanical stimulus and also maintain their viability and physiological properties during experiments. Hence, an effective medium for dynamic cell culture should be (1) anti-cell coagulation, (2) anti-bacterial, (3) PH-invariant, (4) free of bubbles, (5) as close as possible to physiological conditions (e.g. osmolarity and pH), and (6) glucose-rich (ATP source). To meet all these requirements, a customized DMEM-based culture medium was prepared.

To avoid bubble formation and pH fluctuation in the microfluidic channels, a bicarbonate-free version of Dulbecco's Modified Eagle Medium (DMEM-D 1152) with raised glucose level (4500 mg/L, 25 mM) and 25 mM HEPES, compared to regular DMEM (1000 mg/L glucose, no HEPES), was used. The high glucose level was important for maintaining physiological homeostasis of the maturing cells. The initial osmolarity of the DMEM buffer was only ˜210 mosmol/kg due to the absence of sodium bicarbonate. To bring it up to match normal plasma osmolarity (˜290-300 mosmol/kg), an additional 122 mg of glucose and 40 mg of sodium chloride was added to each 25 ml of such medium. Just prior to the introduction of the medium into the microfluidic system, 4% Fetal Bovine Serum (Gemini #900108) and 1% penicillin/streptomycin (Sigma #P4333) were added to the buffer, followed by filtration through a 0.22 μm syringe filter. The solution was de-gassed under vacuum for about 1 hour.

To verify the effectiveness of the customized culture medium, red blood cells were statically cultured in this medium for 48 hours at 37° C. and 5% CO₂ in an Eppendorf tube. This medium was also tested with reticulocytes at 4, 20 (room temperature), and 37° C. over the course of 48 hours. The effects were quantified by the change in sphericity values and visualized by the morphological change observed under a microscope (60×). Constant sphericity and non-altered morphology (round and smooth in contour) was expected to be seen in normal RBC samples for an effective medium.

Enrichment of CD71+ Reticulocyte from Whole Blood

Twenty milliliters of fresh venous blood was withdrawn from donors in the morning (˜9 am) and was spun down at 400 g at 4° C. for 8 mins. The top plasma layer was extracted and placed into a 15-ml Falcon centrifuge tube; on average, about 10 ml of plasma was collected from 20 ml of whole blood. The volume of extracted plasma was replaced by 1% BSA buffer (bovine serum albumin, cat #12659, Calbiochem). A 3-cm high cellulose (a-cellulose and microcrystallin cellulose, Sigmacell Type SO) column bed was packed in a 30-ml syringe tube that was inserted into the neck of a round-bottomed flask; a filter paper was placed on the bottom of the syringe tube to prevent the leakage of cellulose powder. This cellulose column was primed with ˜10 ml of 1% BSA buffer while refrigerated. Once the dripping was noticeably slowed down, buffer collected in the flask was emptied. Six milliliters of the plasma-free whole blood suspended in 1% BSA was poured into this moist cellulose column bed and refrigerated (4° C.). The process of blood filtration removed leukocytes from whole blood. This process was completely gravity-driven and took ˜1 hour to obtain 3 ml of filtered blood. This step is vital because leukocytes have high CD 71 expression that take up the anti-CD71 beads and therefore weaken the efficiency of reticulocyte isolation.

Collected leukocyte-free blood was diluted 1:1 with 0.5% BSA buffer and aliquoted into 1.5 ml/tube (usually 4 tubes, 6 mL in total), and then 50 μL (˜2×107 particles) of BioMag anti-human CD71 coated beads (˜1.5 μm in diameter, Bangs Laboratories, Inc.) were added to each tube. These loaded tubes were wrapped with aluminum foil and incubated on a rotator at 4° C. for 1-2 hours to allow complete binding of anti-CD71 beads to CD71 on the reticulocyte membrane. After the incubation, the tubes were placed in a rack with a cobalt-samarium magnet mounted along the side of the tube (Magnetic Particle Concentrator; Dynal). While on the magnetic rack, CD71+ reticulocytes linked to the beads were withheld by the magnetic field whereas the free CD71− cells (e.g. erythrocytes) were in the supernate. The supernate was carefully removed and replaced by 0.5% BSA buffer, followed by gentle agitation with a pipettor to break up beads clumps. For each wash, the bead-cell mixture was left to stand on the rack for at least 3 mins to allow as many beads to get to the magnet as possible; the wash step was repeated three times or until no blood remainder was seen in the supernate. After the last wash, beads were resuspended with 1 ml of 0.5% BSA buffer and transferred to a new 1.5-ml eppendorf tube. Recombinant human CD71 (Transferrin R, Cata #2474-TR, RnDSystems, Inc.) was added to the previously collected plasma (4 μg/5 ml plasma) to boost CD71 activity of the plasma. Then 0.5 ml of this plasma was added to the washed bead-cell mixture and made up to a total volume of 1.5 ml in each tube. Samples were then placed back on the rotator at 4° C. for 1-2 hours. During this period, the excess concentration of CD71 in the plasma was able to compete with the CD71 receptors on the reticulocytes for the anti-CD71 sites on the beads, resulting in the release of reticulocytes from the beads. Lastly, the reticulocytes were isolated from the beads on the magnetic rack, where beads were withheld by the magnetic field again, and the bead-free suspension was carefully withdrawn and spun down at 300 g for 5 mins to collect pellets, which contained nearly pure CD71+ reticulocytes. Beads were discarded. Reticulocytes were resuspended with the customized DMEM-based culture medium and saved at 4° C. for the later microfluidic testing.

Syringe Pump Setup

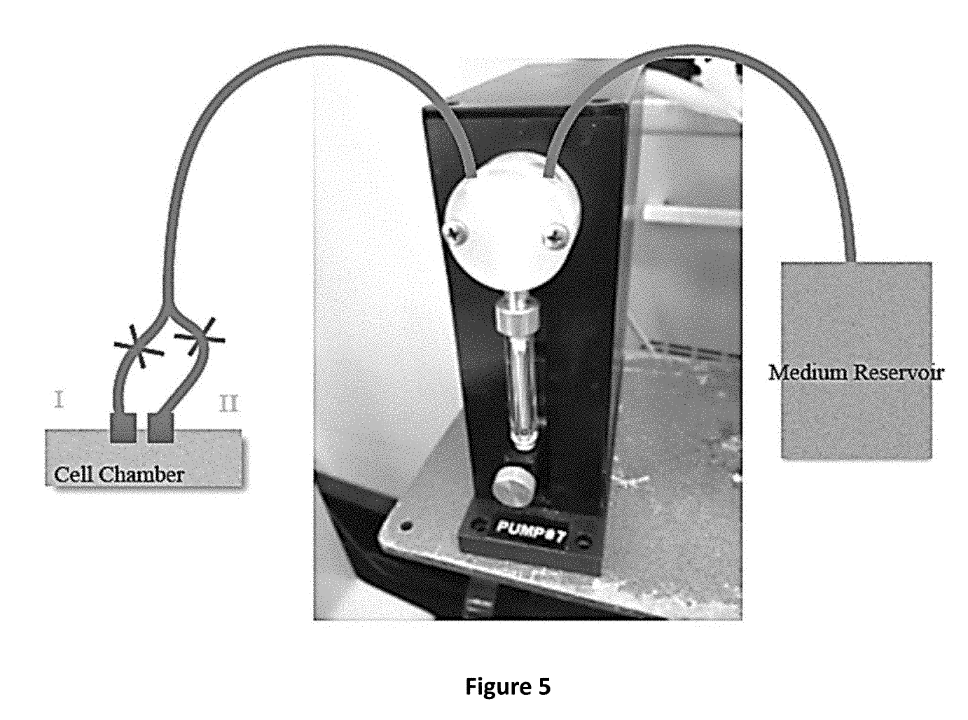

A stepper motor driven pump with a 50-μL glass syringe (C3000 syringe pump; TriContinent Scientific) was used to provide predictable flow and pressure control to the microfluidic system. It had two ports, one connected to the medium reservoir and the other linked to the bioreactor through a Y-shaped PTFE tubing, the split ends of which were plugged into the 1/16″ female luers on the polycarbonate top-piece (FIG. 5). Prior to hooking the bioreactor to the pump, culture medium was flushed through the tubing and glass syringe several times to void air and coat the inner surface.

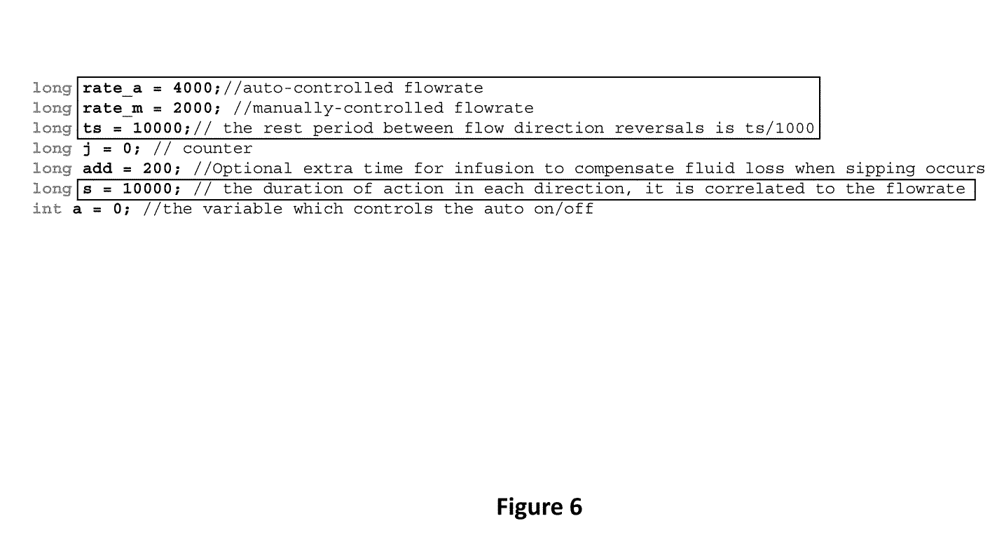

The Arduino micro-controller system was programmed to drive the fluid at desired flow rates in the range between 25-30 μL/min for 30-60 s, with 10 s of resting period between flow reversals. A portion of the Arduino interface is shown in FIG. 6, wherein four programmable parameters are used for controlling the flow rate at automatic mode (rate_a) and manual mode (rate_m), the resting period between flow reversal (ts), and how long the flow persists before reversing (s). The last parameter s is correlated to rate_a; more specifically, for example, s=8000 is equivalent to 20 sec of flow duration when rate_a is 3000, whereas it is 24 sec when rate_a is 4000. The manually-controlled flow rate (rate_m) was manipulated to serve different purposes during the experiment. For example, when culture medium was initially introduced to the cell chamber, the rate_m was set at 500 (˜150 μL/min) to quickly pump the medium through the bottom channel, and upon starting to push medium vertically across the filters, the value of rate_m was doubled (the flow rate decreased to ˜70 μL/min) to avoid breaking the silicon filters. In most of the experiments, the parameter rate_a and s were set as 4000 and 10000, respectively, to produce a flow rate of ˜26 μL/min for ˜30 seconds, and flow reversed every 10 seconds (t5=10,000).

Microfluidic Testing with Cells

Once the functional microfluidic device was made, the feasible source of pressure driven flow and flow parameters were identified, the isolation of adequate numbers of cell of interest was successfully reproduced, and the effective culture medium was composed, the microfluidic system was ready to be tested. Although, up to this point, the microfluidic device was confirmed to be able to drive cells to traverse narrow slits under a tunable pressure without cell loss over time, there was a need to examine whether or not this microfluidic system was harmless to viable cells. Therefore, the morphology and deformability of cells were monitored and measured after being “massaged” in the microfluidic device over a course of 2-6 hours.

The finished microfluidic assembly was first exposed under ultraviolet/ozone treatment for 10 minutes to yield negatively charged and thus hydrophilic filter surfaces. Then, one of the split inlets (I, FIG. 7) was clamped off and the other one (II, FIG. 7) was connected to one of the luer connectors (b, FIG. 7) on the polycarbonate enclosure. The syringe pump was activated to infuse medium into the microfluidic chamber at ˜150 μL/min. The fluid filled up the flow channel below the bottom barrier filter quickly and came out from the other connector (a, FIG. 7) due to the lower resistance in this pathway compared to moving vertically through the filters. Once the fluid appeared at the mouth of the open connector a, the inlet I was ‘un-clamped’, and plugged into this connector. As two ports were plugged, flow was forced to cross the filters and fill up the top compartment (˜25 μL). It should be noted that the flow rate was lowered to ˜70 μL/min for this step so that the fluidic pressure would not burst the fragile filters.

Air bubbles are among the most recurring issues in microfluidics because they are very difficult to remove and can compromise the applied pressure and flow rate. In this work, many precautions were taken to prevent air bubbles. First, the ultraviolet/ozone treatment allowed silicon filters to become more hydrophilic so that the chamber could be entirely filled with the degassed culture medium. Second, medium was first introduced through one port on the chamber at an elevated flow rate while the other port was open to the atmosphere; this step forced fluid to only fill the bottom channel, therefore any bubbles that were created in this step could be pushed out from the open port. When both ports were connected to the syringe pump, flow rate was decreased by half to fill up the cell chamber, and stopped when fluid appeared at the top surface of the open chamber above the filters. Lastly, the medium was given ˜10 mins for fully coating the walls of the open chamber, as well as for allowing the medium to saturate the multilayered micro-structure to fill up all possible voids between layers.

One microliter of packed cells was resuspended into 0.5 ml of DMEM-based microfluidic culture medium, ˜50 μl of which was fed to the microfluidic chamber from the top open well. The syringe pump was activated for fluid withdrawal, which pulled cells down across the upper slit filter, until most of the cell population landed on the lower 0.5-μm porous filter. The pump then paused for 10 s, followed by a reversed flow that pushed cells through the slit filter to the open well.

In this work, three experiments were conducted using the latest version of the microfluidic device. Magnetically-isolated CD71+ reticulocytes were subjected to the periodic ‘massage’ once per minute for 4 hours or once every 30 seconds for 2 hours, thus individual cells repeatedly traversed the slits approximately 240 times in each experiment. Measurements of cellular sphericity were obtained from statically cultured (at 4° C. and/or room temperature) and dynamically cultured (2-4 hours at room temperature) reticulocyte samples.

Measurement of Cell Sphericity

To quantify the effect of in-vitro mechanical stimulation on the development of red blood cell deformability, measurements were used to calculate for cell sphericity (S), a dimensionless quantity containing the ratio of cellular volume to the two-thirds power divided by the membrane area. Note that the maximum value of the sphericity is 1.0, which represents a perfect (undeformable) sphere. The lower the value of the sphericity, the larger is the “excess” area of the cell, and the more deformable the cell becomes:

$$S = \frac{4 π}{{( 4 / 3 π )}^{2 / 3}} \cdot \frac{{V}^{2 / 3}}{A}$$

where S is sphericity, V is cell volume, and A is cell membrane surface area.

Cells were tested before and after treating CD71+ reticulocytes in a microfluidic device for 2-4 hours, and comparing the results with fresh normal red blood cells and control cells kept under the same conditions but not subjected to passage through the micro-slit filter.

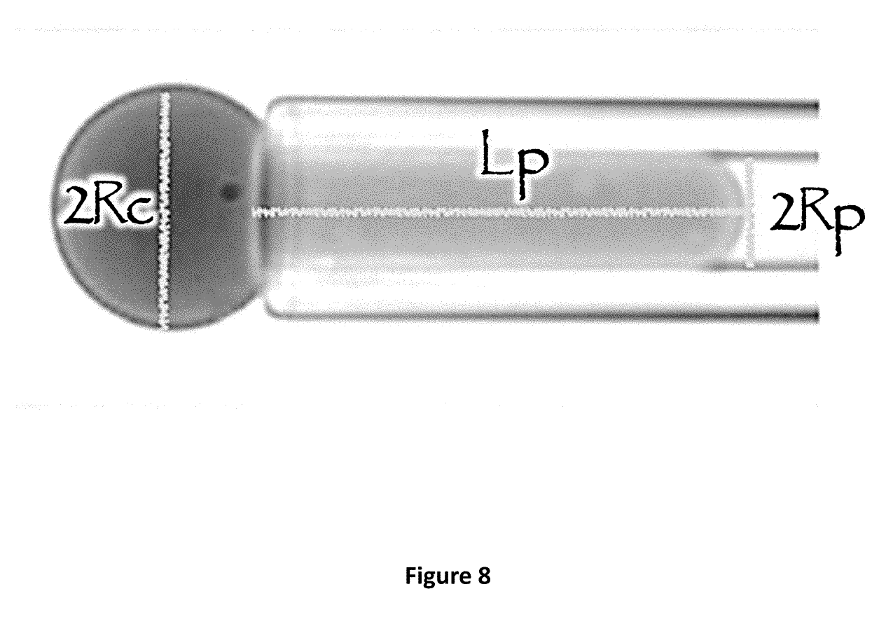

The measurements for cellular volume (V) and membrane area (A) were obtained using micropipettes as described by (Waugh R E et al., Experimental hematology 41.4 (2013): 398-408). Cells were aspirated into a tapered glass micropipette at a pressure of ˜1,500 Pa (15 cm H2O) (FIG. 8). Care was taken to ensure that the membrane was fully extended into the micropipette without fold or crease. About 50 cells were randomly picked and aspirated for each sample.

The cell area A and the cell volume V were calculated from the measurements of the outer cell radius, R_{c}, the inner radius of micropipette, Rₚ, and the length of the projection in the pipette, Lₚ, using the following equations:

$$A = 2 π {R}_{c} ( {R}_{c} + \sqrt{{R}_{c}^{2} - {R}_{p}^{2}} ) + 2 π {R}_{p} {L}_{p}$$

$$V = \frac{2 π}{3} [ {R}_{c}^{3} + ( {R}_{c}^{2} + \frac{{R}_{p}^{2}}{2} ) \sqrt{{R}_{c}^{2} - {R}_{p}^{2}} + {R}_{p}^{3} ] + π {R}_{p}^{2} ( {L}_{p} - {R}_{p} )$$

The results are now described.

Microfluidic Culture Medium Validation

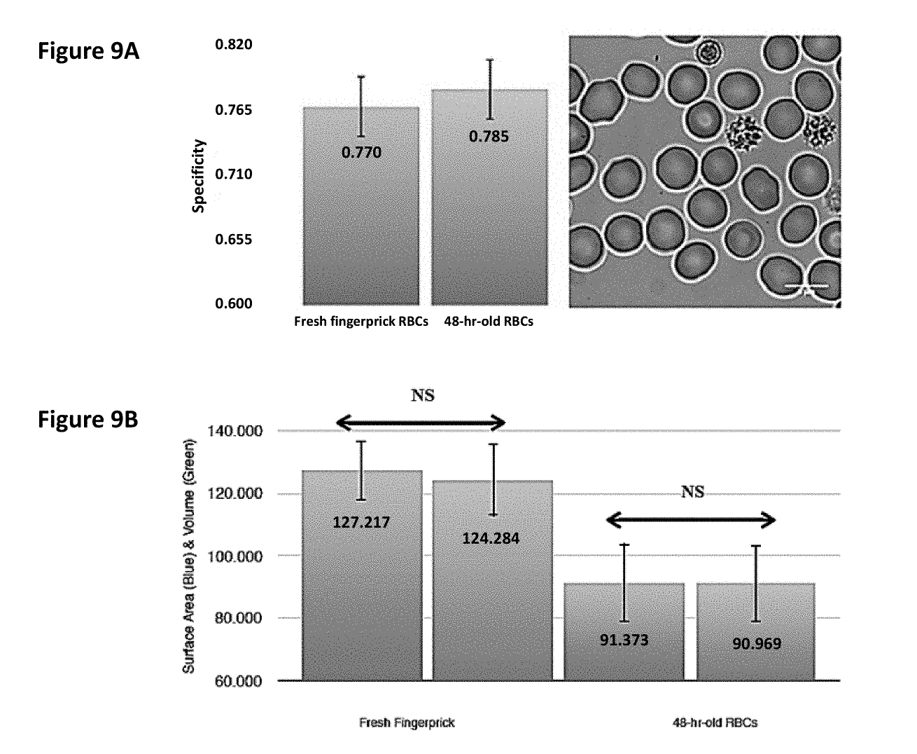

To assess the effectiveness of the microfluidic culture medium, finger-prick samples were statically cultured in this medium for 48 hours at 37° C. and 5% CO2 in Eppendorf tubes. This medium was also tested with reticulocytes at 4, 20 (room temperature), and 37° C. over the course of 48 hours. The effects were identified by the change in sphericity values and qualitatively by morphological changes observed under a microscope (60×). As illustrated in FIG. 9A and FIG. 9B, the mean sphericity values of 48-hour-old RBCs and freshly-drawn RBCs show no significant difference (>50 cells/sample).

It is clear that neither the dimensional parameters nor the morphology of RBCs exhibit significant difference after 48 hours in the culture medium. Thus, the customized DMEM medium, which is anti-bacterial and anti-coagulating and has physiological osmolarity and pH, was suitable for maintaining cell culture over extended times (at least for 48 hours). RBCs stored in such medium were also measured before and after short room-temperature static cultures (-4-6 hours) as a control when microfluidic treated reticulocytes were evaluated, no noticeable change in sphericity or morphology were observed (data not shown).

Assessment of Viability of Reticulocytes Stored at Different Temperatures

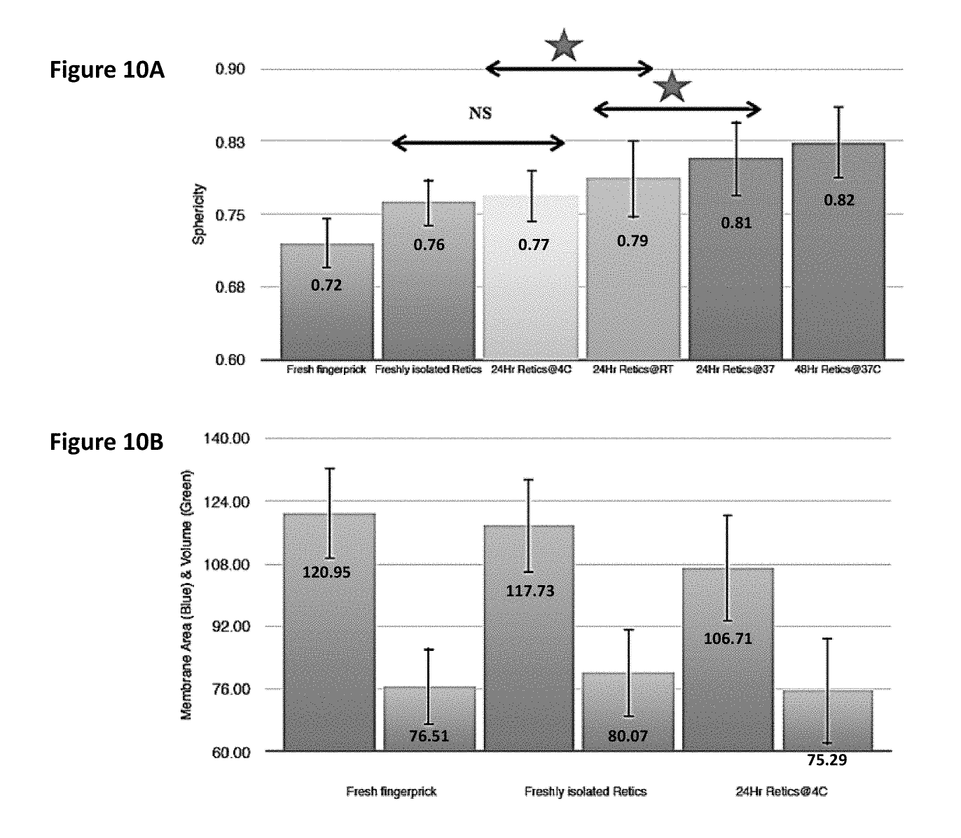

It was demonstrated in prior studies that reticulocytes do not mature at 4° C. in vitro for 2 weeks after they were drawn from animals (Golenda C F et al., Proceedings of the National Academy of Sciences 94.13 (1997): 6786-6791). The results were generally in accordance with the reported observation. As illustrated in FIG. 10A, the mean sphericity of freshly prepared reticulocytes (green bar) was not significantly different from that of 24-hour-old reticulocytes incubated at 4° C. (yellow). On the contrary, the 24-hour-old reticulocytes that had been incubated at room temperature (orange bar) and at 37° C. (red bar) indicated greater sphericity. The sample incubated at 37° C. for 48 hours (purple) exhibits the highest sphericity.

To further examine the mechanism underlying the spontaneous increase in sphericity in static culture over the course of 24 hours at both room temperature and body temperature, this property was dissected into two factors—the surface area and the cellular volume, from which the sphericity values were calculated. As suggested in FIG. 10B, the freshly isolated reticulocytes possessed similar amount of membrane area and greater cellular volume compared to the finger-prick cells, and after 24-hour static incubation at 37° C., they lost a considerable amount of volume and membrane area, yielding a similar cellular volume as the finger-prick sample and an average of ˜12% less membrane area.

This further analysis for the data presented in FIG. 10A leads to the inference that the isolated reticulocytes underwent the removal of cellular components in the static culture, and this was accompanied by an abnormally greater membrane area loss, resulting in less excess membrane area in the end-product compared to normal erythrocytes.

It should be noted that the sphericity value of the freshly isolated reticulocyte sample was higher than that of the whole blood samples (FIG. 10A, second and first bars from the left, respectively). The most likely explanation for this is that the measurement of the fresh reticulocyte sample was actually performed at 7 or 8 h after cells were withdrawn from the donor because of the length of the protocol used to isolate them. When the isolation protocol was shortened to 4 hours and the cells were carefully maintained at ˜4° C. during the entire isolation, the sphericity of isolated reticulocytes was found to be slightly lower than that of whole blood.

In summary, membrane remodeling of CD71+ reticulocytes, reflected by a noticeable membrane loss, can occur in static culture at room temperature and 37° C.

This membrane loss was observed to be suppressed during the 24-hour incubation at 4° C. No noticeable change in the mean sphericity of natural erythrocytes was observed over 48 hours at room temperature and 37° C.

Microfluidic Testing with Whole Blood

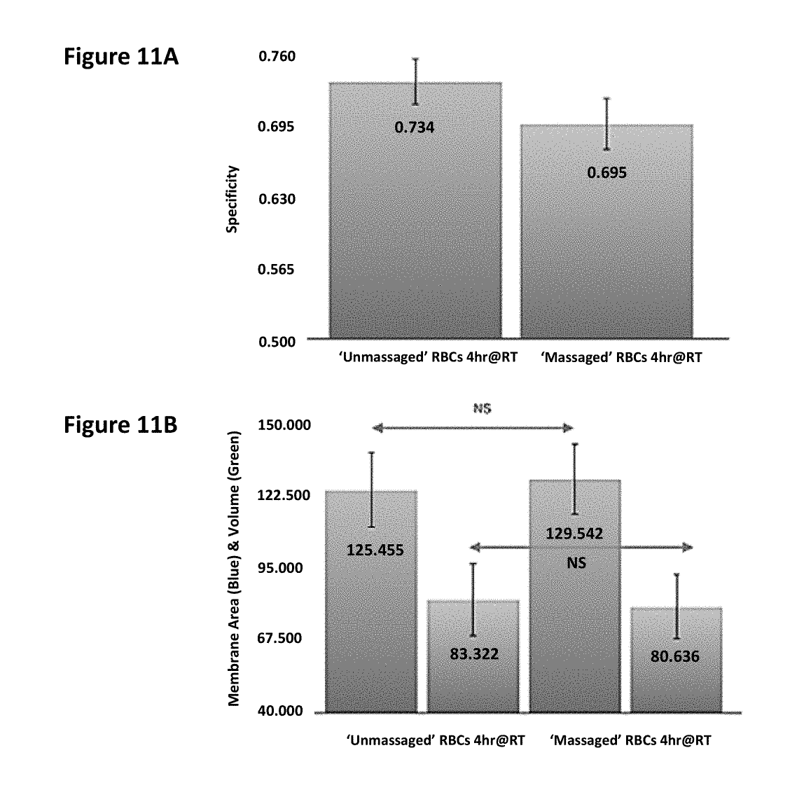

In the human circulation, mature erythrocytes are removed from the circulation after ˜120 days. These senescent cells are trapped and removed from circulation by the spleen, whereas the healthy cells pass through the splenic sinus slits and rejoin the circulation. The microfluidic device was thus tested to ensure it would not compromise the viability and functionality of normal erythrocytes. The cellular dimensions of “massaged” and “unmassaged” cells from whole blood were measured after four hours of treatment. The morphology of the RBCs was normal after treatment (not shown). A significant decrease in sphericity with microfluidic treatment was observed (FIG. 11A), but when the volume and area data were compared, the differences between samples was no longer significant (FIG. 11B). This most likely reflects greater variability in the calculated values for area and volume because errors in the measurement of R_{c} enter the area calculation as the square and the volume calculation as the cube, but tend to cancel out when calculating the sphericity. The decrease in sphericity could reflect an adaptation of the cells to tighter confines in the microfluidic device than the cells encounter in vivo. It seemed clear however that significant physical damage to the cells did not occur over the course of 4 hours (˜250 passages).

Microfluidic Testing with Isolated CD71+Reticulocytes

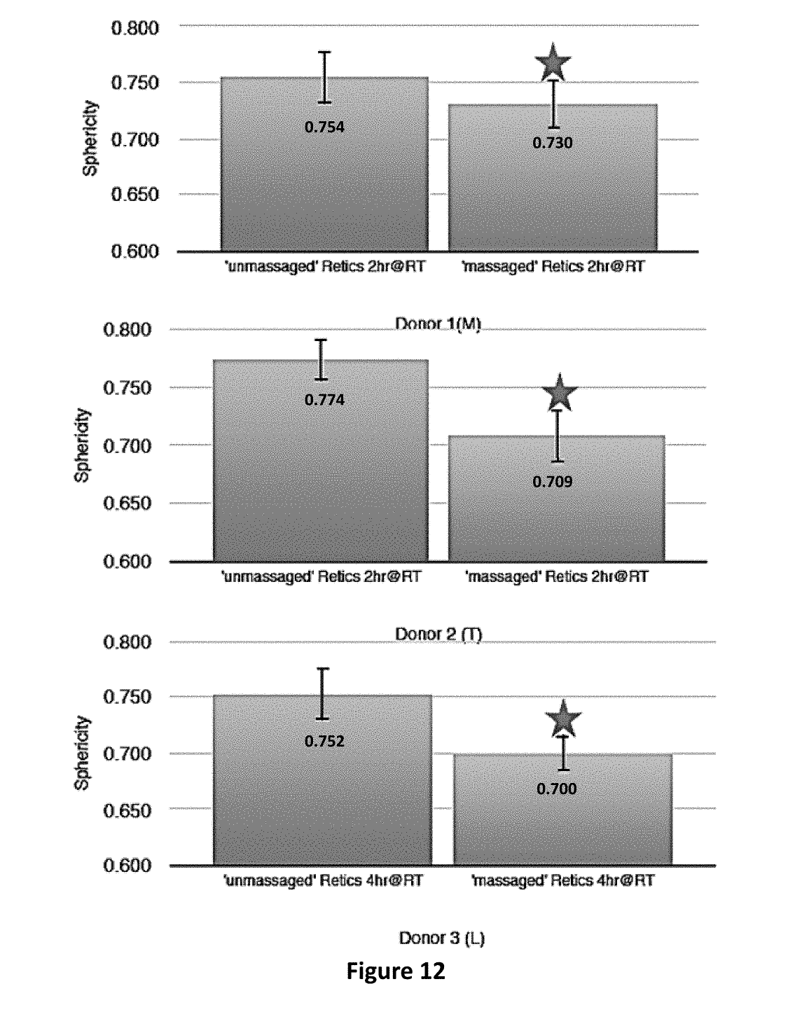

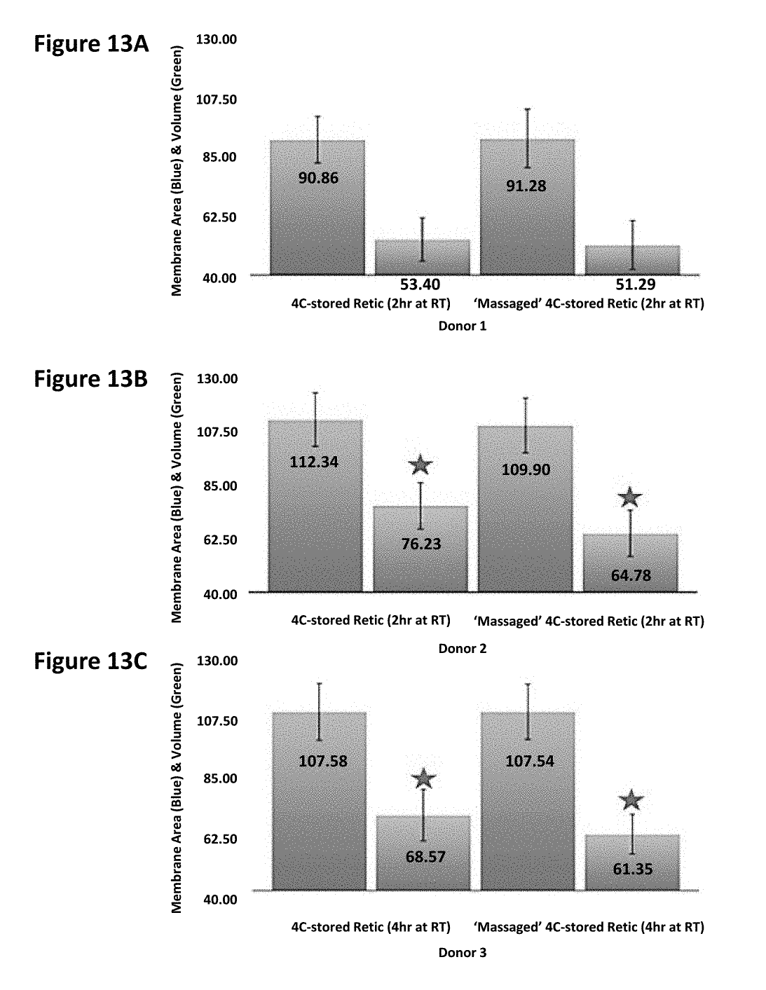

Up to this point, the device was successful in meeting the study requirements in managing cells under programmable fluidic conditions for up to 4 hours of treatment. Nevertheless, nearly all the cells turned into echinocytes in 6 hours in the microfluidic devices. Therefore, microfluidic tests with isolated CD71+ reticulocytes were carried out by setting the periodic cell passages through the slit filter once per minute for 4 hours or once every 30 seconds for 2 hours. Overall, individual cells repeatedly traversed the slits approximately 240 times in each experiment. Five experiments were conducted using CD71+ reticulocytes obtained from venous blood donated by five healthy individuals. Isolated reticulocytes were stored in aliquots and refrigerated (4° C.) for ˜12 hours before loading into the microfluidic device. The sphericity values of cells from four different conditions were tested: reticulocytes before and after microfluidic treatment, reticulocytes left in the same conditions but without mechanical stimulation, and reticulocytes kept refrigerated until the other measurements were completed. The results of the reticulocytes before and after microfluidic treatments shown in FIG. 12 revealed the effect of the microfluidic treatment on decreasing the cellular sphericity, and FIG. 13A through FIG. 13C further confirmed that this decreased V/S ratio was mainly due to the reduction of cellular volume (Three examples are shown).

It should be noted that, although the experimental duration was 2 hours for the first two experiments and 4 hours for the last one, the total numbers of passages of the cells through the slits were approximately the same (˜240 times), and the overall time that they had been exposed to room temperature before measurement was also similar.

Although all three experiments show a significant decrease in sphericity and the membrane areas were retained after the microfluidic treatments, the expected considerable volume reductions were only observed in the last two experiments—there was a slight decrease in volume observed for the first experiment. Nevertheless, the overall trends for decreased cellular volumes and constant membrane areas were fairly consistent for all experiments. Based on the observations depicted in FIG. 11A, FIG. 11B, and FIG. 13A through FIG. 13C, it was concluded that the mechanical stress enhanced membrane area retention during the transition from reticulocytes to deformable erythrocytes. The surface areas and cellular volumes of whole blood cells, newly isolated reticulocytes, and overnight refrigerated reticulocytes of all three donors were also measured and the sphericity values were calculated from them.

Overall, these results demonstrate that the repeated passages through narrow slits, which exert mechanical stress on cell membrane, likely accounted for the maintenance of sphericity observed during the reticulocytes transition into deformable erythrocytes. The decreased sphericity observed is believed to contribute to the acquisition of deformability of mature erythrocytes and is critical for them to survive in the microvasculature during the ˜120 days of travel in the circulation.

Existing Approaches for Erythrocyte Production Fall Short In Terms of Reticulocyte Maturation

Over the past decades, many labs have made attempts to achieve the ex vivo largescale production of erythrocytes. Although the final products from these reported methods are not practical for clinical transfusion, the technique of ex vivo erythropoiesis has been improving and evolving. The importance of neighboring stromal cells and macrophages, appropriate stem cell sources, and several exogenous factors (EPO, SCF, cortisol, etc.) in human erythropoiesis have been commonly recognized. Building on previous findings, Fujimi et. al. produced a total of 11.8 units, the highest yield ever reported, of enucleated near-to-maturity red blood cells from one unit of human cord blood using a four-phase culture system (Fujimi A et al., International journal of hematology 87.4 (2008): 339-350). Despite the excellent work that has been done to reproduce the biochemical and cellular microenvironment in the bone marrow where immature erythroid cells reside and differentiate, this is not the whole story of erythropoiesis. Adult erythroid cells continue their journey toward complete maturation into erythrocytes during the first 24-48 hours after their release into the circulation. The mechanism underlying this final step of erythrocyte maturation, where erythrocytes acquire proper cellular deformability and stability and remove redundant cellular components when they pass through the sinusoids in hematopoietic organs (Gronowicz G et al., Journal of cell science 71.1 (1984): 177-197), is still unclear.

Evidence that Reticulocytes Do No Mature Properly in Static Culture

One thing that does seem to be clear is that this late stage remodeling is disregulated when reticulocytes are allowed to mature in static culture. A collaborator who has been studying how a cell decides to either self-renew or to differentiate found that >80% of murine-derived ESREs undergoing induced maturation in maturation media were enucleated after three days, and also observed that the cells exhibited morphological characteristics typical of orthochromatic erythroblasts or reticulocytes. Nevertheless, a subset of these cells had small vacuoles and irregularities in contour compared to peripheral blood erythrocytes. Recently, Mankelow, et al., who tested numerous reported blood cultivation methods and found that 5% to 10% of in vitro-produced reticulocytes from all cultures have an external PS-positive inside-out vesicle (Mankelow T J et al., Blood 126.15 (2015): 1831-1834). An elevated number of PS-positive immature erythrocytes in the blood can be problematic as they can lead to a hypercoagulable state through increased thrombin generation and associated platelet activation (Ataga K I et al., British journal of haematology 139.1 (2007): 3-13). Clinically, it is observed that patients whose spleens are absent have increased numbers of erythrocytes containing autophagic vacuoles that are also PS-positive (Griffiths R E et al., Blood 119.26 (2012): 6296-6306). The present results examining the progression of circulating reticulocyte maturation in vitro show a progressive increase in sphericity with time in static culture (FIG. 10A). All of these results point to a role for mechanical stimulus for proper late-stage reticulocyte maturation.

Slits are Favorable Over Pores

Several generations of microfluidic prototypes were designed and constructed. These devices were first tested with whole blood, before infusing reticulocytes into them. Cell damage, evidenced by the formation of echinocytes, had been observed before the upper filter was switched from the 1-μm diameter pores (Format B) to the 1.5-μm wide slits (Format C). Most of the infused cells became echinocytic in less than 2 hours. It should be noted that cells encountered large stress when they squeeze through the 1-μm diameter pores; moreover, many cells were simultaneously captured and overly stretched by two pores as the space between them was shorter than the diameter of average erythrocytes. It has been documented that normal RBCs produce a tendency to form echinocytic spicules when the exterior surface area of the bilayer membrane is larger than the cytoplasmic surface area (Sheetz M P et al., Proceedings of the National Academy of Sciences 71.11 (1974): 4457-4461). Both biochemical agents, including anionic amphipaths, high salt, high pH, and mechanical forces have been proposed to play roles in expanding the outer leaflet relative to the inner one (Lim H W G et al., Proceedings of the National Academy of Sciences 99.26 (2002): 16766-16769). Generally speaking, RBCs tend to maintain their low-energy shapes when they encounter mechanical stress. Creating echinocytic spicules involves a balance of elastic energies due to the tendency of the bilayer to deform outward, and the deformation of the membrane skeleton that is required for shape changes (Waugh R E et al., Journal of Laboratory and Clinical Medicine 129.5 (1997): 527-535). During passage through the 1-μm diameter pores, the mechanical stress applied to the cells might be larger than in-vivo conditions and therefore led to a substantial asymmetry between leaflets; as a result, the observed spicules were small and numerous and eventually irreversibly bud off.

To enhance the cell viability in the microfluidic device, the 1-μm microporous filter was replaced with the current slit filter. However, although the stress resulting from passage of the 1.5-μm wide slits was believed to be less harsh to cell membranes, cells still turned into echinocytes after 6 hours of the microfluidic treatment. This is believed to be the result of the cells being captured against the barrier filter used in the current design. In order to retrieve sufficient number of non-echinocytic cells after experiments, cells were set to pass through slits either once per minute for 4 hours or once every 30 seconds for 2 hours in all experiments. It is expected that elimination of the barrier filter should enable the device to be used for much longer periods of exposure.

The Importance of Sphericity in Red Cell Viability

The ratio of cell volume to membrane area is captured in the dimensionless quantity called the sphericity. It is a critical determinant of the ability of red cells to survive in the vasculature. This was most clearly shown in a study that examined the consequences of removing membrane area from mouse red blood cells and then testing the consequences of the surface area loss on the ability of the cells to survive when reinfused into a recipient mouse. The study showed that erythrocytes with sphericity more than 5% above the normal range were removed from the circulation or modified in vivo to fall within 5% of normal (Murdock RC et al., American Journal of Physiology-Cell Physiology 279.4 (2000): C970-C980). Thus, a critical outcome for red cell maturation is the acquisition of a sphericity that is sufficiently low for the cell to survive in the vasculature. The sphericity of a fully mature healthy erythrocyte was measured by interference microscopy to be 0.79 with a standard deviation (SD) of 0.05 at room temperature (Fung Y C et al., Biorheology 18.3-6 (1981): 369), and by micropipette to be 0.73 with a SD of 0.02 (Waugh R E et al., Blood 79.5 (1992): 1351-1358). The micro-slits used in the current study were designed to achieve these values. Based on the reported values of surface area and cellular volume of human RBCs, 135±10 μm² and 93±12 μm³ , respectively, measured by micropipette aspiration (Waugh R E et al., Blood 79.5 (1992): 1351-1358), the width of the narrowest slit through which each RBC could pass was also calculated. The slits employed in this work were approximately 1.5-μm wide; these slits might be a little too narrow for reticulocytes to pass through, implying that the narrow slits might be partially responsible for the echinocytic transformation observed over long experiment (>6 hours) and also explain the significant decrease in the ‘massaged’ reticulocyte sphericity which was even lower than that of natural RBCs. On the other hand, when the width of slit is given and either the volume or surface area is confidently measured, the cellular sphericity required for slit passage can also be calculated and then compared with the experimental outcomes. The expected sphericity that enables cells to flexibly traverse the 1.5-μm wide slits in our microfluidic device has been predicted to be 0.72, 0.68, and 0.69 for donor 1 to 3, respectively, while the experimental measurements were actually 2.9 ±1.5% higher than these predicted values. Two assumptions underlying these calculations were that, the measurements for the cellular volume and surface area were accurate and the width of the slits integrated in the microfluidic devices was 1.5 μm. The disagreement between the experimental and expected sphericities might be a result of either measurement errors or the underestimate of the actual width of slits—the slits were probably not as small as expected.

Mechanisms by which Mechanical Stress Reduce Area Loss and Decrease Cell Volume

Although there could be multiple contributing factors to the acquisition of proper sphericity and consequent maturation into an erythrocyte, it was hypothesized that mechanical stretch resulting from the passage of reticulocytes through slits like those in the walls of splenic sinuses, the narrowest of which are only 1-2 μm wide, can help regulate the process of membrane remodeling. The present results show that reticulocytes do in fact become more deformable with the microfluidic treatment (FIG. 12). This enhancement in deformability, reflected by the decreased sphericity (V/S ratio), appears to be a consequence of a loss of cellular volume and the preservation of membrane area (FIG. 13A through FIG. 13C). When a cell gets stuck in an aperture, the resulting mechanical forces act to either increase the cell surface area (as a result of the build up of membrane tension) or reduce its volume in order to pass through. This proposed mechanical adaptation can be attributed to effects of mechanical tensions. One speculation is that mechanical tensions play a role in regulating the size of exocytosed microparticles and autophagic vacuoles by pulling attached vesicles and other attached membrane structures back onto the cell surface, making the shedding vesicles as small as possible. These tensions could also activate volume regulatory channels in the membrane. For example, mechanical stretch is known to activate Ca2+ influx in red cells, and this leads to activation of K+ channels, allowing K+ efflux and cellular dehydration (Dyrda A et al., PloS one 5.2 (2010): e9447). This mechanically-induced Ca2+-dependent K+ leakage, often noted in the literature as the “Gardos phenomenon,” could also contribute to the reduction in cell volume noted in the ‘massaged’ cell samples, as illustrated in FIG. 13A through FIG. 13C.

Implications of Results

Intriguingly, after 4 hours of traversing the slits, although the changes in volume and membrane area were not statistically significant (FIG. 11B), the ‘massaged’ RBCs had lower sphericity than the ‘unmassaged’ RBCs (FIG. 11A). In other words, mature RBCs with well-developed deformability became even more deformable after the microfluidic treatment. Moreover micro-slits used here might be too narrow to reproduce or maintain a physiologically comparable sphericity. Taken together, these considerations suggest that RBC's adapt themselves to the 1.5 μm wide slits (which mimic the narrowest apertures cells encounter in vivo) by reducing their volume, most likely through mechanically induced potassium leakage and the accompanying water loss, so that they could more easily traverse the 1.5 μm slits. Measurements of the cellular volume and membrane area were carried out before and after the 2 or 4 hour of microfluidic treatments and the corresponding sphericity values were calculated from these measurements. The results of FIG. 12 coincide with the hypothesis that the sphericity values of reticulocytes decreased with the microfluidic treatments, implying reticulocytes became more deformable after being subjected to periodic mechanical stress. Examination of the area and volume of cells from these samples indicates that the decreased sphericity was largely a result of the decreased volume and constant membrane area. In two out of three cases, the reduction in volume was statistically significant, and in the third, the data were trending in the same direction. This decreased volume could be explained by possible dehydration due to the increased ion loss under stress, as described above.

In addition to the results presented in FIG. 12 and FIG. 13A through FIG. 13C, the sphericity values, membrane areas, and cellular volumes were also measured for the whole blood cells and newly isolated reticulocytes (or overnight refrigerated reticulocytes) for all three experiments. Notice that, the ‘massaged’ reticulocytes not only show lower sphericity than their ‘unmassaged’ counterparts, but also than the fresh whole blood cells from the same donor. This appears to be the result of a noticeable decrease in cellular volume. This result is in line with the previous observation that mature erythrocytes extended their well-developed deformability during the microfluidic treatment to adapt themselves to the silicon slits that are analogous with the narrowest sinus slits in vivo, suggesting that mechanically induced cell dehydration might be a unique way for red blood cells (regardless of their maturity) to adjust themselves to negotiate the in-vivo microvasculature and the present in-vitro simulation.

Altogether, it is evident from the results that mechanical tensions are important factors in the decreased V/S ratio that has been recognized during the transition of reticulocytes into more deformable erythrocytes. Two possible mechanically-induced mechanisms, the retention of surface area by pulling as much lipid area from the budding vesicles back onto the cell membrane as possible and the reduction of cellular volume through water loss that follows the Ca²⁻-dependent K⁺ leakage, are discussed above.

The current work indicates a promising “mechanical” avenue to decrease the volume-to-area ratio of reticulocytes in vitro. This ratio is known to be inversely related to the cellular deformability, and it appears that this approach should facilitate their maturation into deformable erythrocytes.

The disclosures of each and every patent, patent application, and publication cited herein are hereby incorporated herein by reference in their entirety. While this disclosure has been disclosed with reference to specific embodiments, it is apparent that other embodiments and variations of the presently described devices and methods may be devised by others skilled in the art without departing from the true spirit and scope of the present disclosure. The appended claims are intended to be construed to include all such embodiments and equivalent variations.

1. A micro-slit filter device comprising at least one micro-slit channel, each micro-slit channel comprising two openings connected by a lumen, wherein the two openings and the lumen comprises a width between about 1 and 3 μm.

2. The device of claim 1, wherein the micro-slit openings comprise a height between about 10 and 100 μm.

3. The device of claim 1, wherein the lumen comprises a length between about 10 and 100 μm.

4. The device of claim 1, wherein the micro-slits are spaced apart by a distance between about 5 and 50 μm.

5. The device of claim 1, wherein the micro-slits comprise a taper.

6. The device of claim 1, wherein the device is constructed from a material selected from the group consisting of: plastics, polymers, metals, glass, ceramics, silicon wafers, and polydimethylsiloxane (PDSM).

7. A microfluidic device comprising:
a bottom substrate;
a first layer positioned on top of the bottom substrate, the first layer comprising a fluid channel having at least one fluid port;
a second layer positioned on top of the first layer, the second layer comprising a space fluidly connected to the fluid channel of the first layer;
a third layer positioned on top of the second layer, the third layer comprising a space fluidly connected to the space of the second layer;
a fourth layer positioned on top of the third layer, the fourth layer comprising a space fluidly connected to the space of the third layer;
the micro-slit filter device of claim 1 positioned within the space of the third layer;
a top substrate positioned on top of the fourth layer, the top substrate comprising fluid tubes connected to the fluid ports of the first layer; and
a compressible material surrounding the first layer, the second layer, the third layer, and the fourth layer, wherein the compressible material is sandwiched between the bottom substrate and the top substrate.

8. The device of claim 7, wherein the fourth layer comprises a compressible material.

9. The device of claim 7, wherein the top substrate further comprises an open well fluidly connected to the space of the fourth layer.

10. The device of claim 7, further comprising between the first layer and the second layer:
a fifth layer positioned on top of the first layer, the second layer comprising a space fluidly connected to the fluid channel of the first layer;
a sixth layer positioned on top of the second layer, the third layer comprising a space fluidly connected to the space of the second layer; and
a barrier filter positioned within the space of the sixth layer;
wherein the barrier filter comprises micro pores 0.5 μm in diameter.

11. The device of claim 7, wherein the device is subjected to ultraviolet/ozone treatment to increase hydrophilicity.

12. A method of mechanically stimulated culturing to produce red blood cells, the method comprising the steps of passing a volume of cell-containing medium through a micro-slit filter device comprising at least one micro-slit channel, each micro-slit channel comprising two openings connected by a lumen, wherein the two openings and the lumen comprises a width between about 1 and 3 μm.

13. The method of claim 12, wherein the cells are whole blood isolated CD71+ reticulocytes, whole blood isolated CD34+ reticulocytes, extensively self-renewing erythroblast (ESRE) derived reticulocytes, mesenchymal stromal cell-derived reticulocytes, bone marrow aspiration-derived reticulocytes, induced pluripotent stem cell-derived reticulocytes, embryonic stem cell-derived reticulocytes, and cord blood-derived CD34+reticulocytes.

14. The method of claim 12, wherein the mechanically stimulated culturing occurs at room temperature.

15. The method of claim 12, wherein the mechanically stimulated culturing occurs at 37° C.

16. The method of claim 12, wherein the micro-slit filter device is incorporated in a microfluidic device comprising:
a bottom substrate;
a first layer positioned on top of the bottom substrate, the first layer comprising a fluid channel having at least one fluid port;
a second layer positioned on top of the first layer, the second layer comprising a space fluidly connected to the fluid channel of the first layer;
a third layer positioned on top of the second layer, the third layer comprising a space fluidly connected to the space of the second layer;
a fourth layer positioned on top of the third layer, the fourth layer comprising a space fluidly connected to the space of the third layer;
a top substrate positioned on top of the fourth layer, the top substrate comprising fluid tubes connected to the fluid ports of the first layer; and
a compressible material surrounding the first layer, the second layer, the third layer, and the fourth layer, wherein the compressible material is sandwiched between the bottom substrate and the top substrate;
such that the micro-slit filter device is positioned within the space of the third layer.

17. The method of claim 12, wherein the cell-containing media is passed through at least one micro-slit filter device in a bioreactor.

18. The method of claim 12, wherein the cell-containing media is passed through the micro-slit filter device at a rate of 25 to 30 μL/min.

19. The method of claim 12, wherein the cell-containing media is passed through the micro-slit filter device over a period of time between about 1 and 7 days.

20. The method of claim 12, wherein the cell-containing media is passed through the micro-slit filter device once to leave the cells to culture within the micro-slit lumen.
